(19) United States
(12) Patent Application Publication
(10) Pub. No.: US 2024/0240042 A1
(43) Pub. Date: Jul. 18, 2024

(54) PHOSPHORESCENT TATTOO INK FORMULATION

(71) Applicants: Dallas Roger Snydmiller, Grande Prairie (CA); Tierra Davies, Grande Prairie (CA); Todd Quesnelle, Grande Prairie (CA); Robert Simmons, Bezanson (CA)

(72) Inventors: Dallas Roger Snydmiller, Grande Prairie (CA); Tierra Davies, Grande Prairie (CA); Todd Quesnelle, Grande Prairie (CA); Robert Simmons, Bezanson (CA)

(21) Appl. No.: 18/155,315

(22) Filed: Jan. 17, 2023

(51) Int. Cl. C09D 11/50; A61K 8/19; A61K 8/34; A61M 37/00; A61Q 1/02

(52) U.S. Cl. CPC C09D 11/50; A61K 8/19; A61K 8/345; A61M 37/0084; A61Q 1/02

(57) ABSTRACT

A tattoo ink formulation that is operable to provide a long persistence of phosphorescence subsequent dermal presentation. The formulation of the present invention includes strontium aluminate as a pigment base wherein the pigment based further includes at least one dopant. In the preferred embodiment of the present invention either europium or dysprosium as employed as dopants wherein the dopants result in the pigment base having a behavior of a photoluminescent phosphor with a long persistence of phosphorescence. The tattoo ink formulation further includes glycerin wherein the glycerin comprises between sixty-five to sixty seven percent of the total volume of the formulation. The formulation further includes approximately one to two percent of ethanol. In a preferred embodiment of the present invention the particle size of the pigment base is one micron and is dermally presented with round shader tattoo needles.

### FIELD OF THE INVENTION

The present invention relates generally to tattooing, more specifically but not by way of limitation, a tattoo ink formulation wherein the tattoo ink of the present invention includes Strontium Aluminate activated with dopants wherein the tattoo ink formulation of the present invention is operable to produce a phosphorescent tattoo.

### BACKGROUND

As is known in the art, tattooing goes back as far as back as Neolithic times. Ancient tattooing was performed for various cultural and religious reasons and utilized primitive tools such as fish bones to pierce the skin and apply ink thereto. The word tattoo is derived from the Tahitian word, tatau, and was introduced into the English language subsequent one of Captain James Cook's expeditions to the South Pacific. Over the past three decades tattooing has become more popular in the United States and the practice has crossed social boundaries and having a tattoo is considered more socially acceptable than in the past. Artists create tattoos by injecting ink into a person's skin. To accomplish this the artist will utilize a needle that punctures skin at a rate of 50 to 3000 times per minute. The needle typically is calibrated to penetrate the skin about a millimeter and deposits a drop of insoluble ink.

One issue with tattooing can be the appearance thereof on darker skin tones or at night. Tattoo inks are manufactured from materials such as but not limited to titanium dioxide. These materials are designed to be permanent and typically have a dark appearance. The dark appearance of the tattoo utilizing traditional tattoo ink can create a tattoo having features that are difficult to see. Whether in low light conditions or on darker skin tones, tattoos and the features thereof are often difficult to visualize.

It is intended within the scope of the present invention to provide a tattoo ink formulation that is operable to be easily seen in low light conditions as well as on darker skin tones.

### SUMMARY OF THE INVENTION

It is the object of the present invention to provide a tattoo ink formulation that is configured to provide visibility to a tattoo created from the tattoo ink formulation of the present invention in low light conditions as well as darker skin tones wherein the tattoo ink formulation of the present invention includes strontium aluminate.

Another object of the present invention is to provide a tattoo ink formulation that is operable to produce a tattoo having increased visibility wherein the formulation of the present invention includes at least one dopant.

A further object of the present invention is to provide a tattoo ink formulation that is configured to provide visibility to a tattoo created from the tattoo ink formulation of the present invention in low light conditions as well as darker skin tones wherein the formulation of the present invention can employ Europium or Dysprosium as dopants.

Still another object of the present invention is to provide a tattoo ink formulation that is operable to produce a tattoo having increased visibility wherein the formulation of the present invention further includes glycerin.

An additional object of the present invention is to provide a tattoo ink formulation that is configured to provide visibility to a tattoo created from the tattoo ink formulation of the present invention in low light conditions as well as darker skin tones wherein the formulation further includes approximately one to two percent ethanol.

Yet a further object of the present invention is to provide a tattoo ink formulation that is operable to produce a tattoo having increased visibility wherein the pigment size of the formulation of the present invention is one micron.

Another object of the present invention is to provide a tattoo ink formulation that is configured to provide visibility to a tattoo created from the tattoo ink formulation of the present invention in low light conditions as well as darker skin tones wherein the formulation is applied to the skin utilizing a round shader tattoo needle.

An alternate object of the present invention is to provide a tattoo ink formulation that is operable to produce a tattoo having increased visibility wherein the tattoo ink formulation includes no more than thirty three percent Strontium Aluminate.

Still a further object of the present invention is to provide a tattoo ink formulation that is configured to provide visibility to a tattoo created from the tattoo ink formulation of the present invention in low light conditions as well as darker skin tones wherein the formulation of the present invention can further include Zinc Sulfide.

A further object of the present invention is to provide a tattoo ink formulation that is operable to produce a tattoo having increased visibility wherein the tattoo ink formulation is mixed at ambient temperature.

An alternative objective of the present invention is to provide a tattoo ink formulation that is configured to provide visibility to a tattoo created from the tattoo ink formulation of the present invention in low light conditions as well as darker skin tones wherein the formulation is stored above ten degrees Celsius subsequent manufacture thereof.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a flowchart diagram of the manufacturing of the tattoo ink formulation of the present invention.

### DETAILED DESCRIPTION

Referring now to the drawings submitted herewith, wherein various elements depicted therein are not necessarily drawn to scale and wherein through the views and figures like elements are referenced with identical reference numerals, there is illustrated a phosphorescent tattoo ink formulation 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

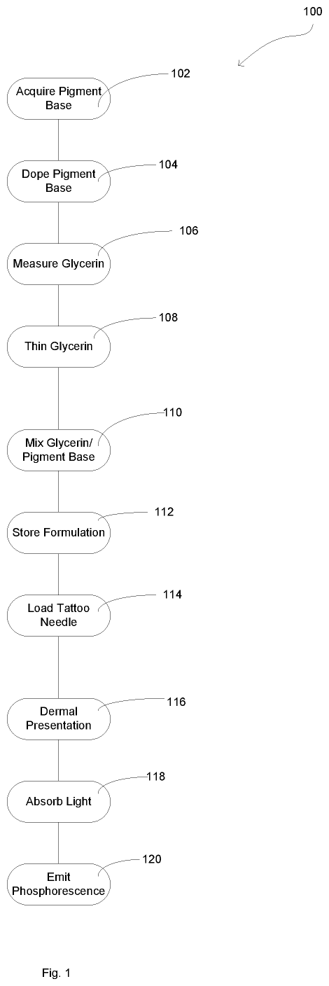

Referring now to the drawings submitted as a part hereof, the phosphorescent tattoo ink formulation 100 is configured to provide a tattoo ink that is more visible on darker skin tones as well as in low light conditions. Illustrated in FIG. 1 herein is an outline of the method of producing the formulation of the present invention. It should be understood within the scope of the present invention that variations thereof are contemplated. In step 102, a pigment base is acquired. The pigment base for the formulation of the present invention is strontium aluminate. Step 104, the pigment base is doped with a dopant in order to achieve the desired appearance of the final tattoo created with the phosphorescent tattoo ink formulation 100. While various dopants could be employed, in the preferred embodiment of the present invention either europium and/or dysprosium(dopants) is utilized to dope the strontium aluminate in order to achieve the desired effect of the phosphorescent tattoo ink formulation 100. Activation of the strontium aluminate with either of the aforementioned dopants will result in the behavior of a photoluminescent phosphor with a long persistence of phosphorescence. In step 106, a desired amount of glycerin is measured. The glycerin is in liquid form and it should be understood within the scope of the present invention that the glycerin will comprise between sixty-five and sixty-seven percent of the total volume of the phosphorescent tattoo ink formulation 100.

Step 108, the glycerin will have a thinner mixed therewith. In order to achieve desired dermal presentation of the phosphorescent tattoo ink formulation 100, the glycerin is thinned with an appropriate material such as but not limited to ethanol. In a preferred embodiment of the present invention, the thinning agent does not comprise more than one to two percent of the total volume of the phosphorescent tattoo ink formulation 100. In step 110, the pigment base and the thinned glycerin are mixed utilizing suitable equipment. The pigment base of the final volume does not exceed more than thirty-three percent of the total volume of the final formulation. Step 112, the formulation of the present invention is stored until ready for use. The storage of the formulation of the present invention should occur at a temperature above ten degrees Celsius. Storage below the aforementioned temperature results in decomposition of the formulation. In step 114, the tattoo ink applicator is loaded with the formulation of the present invention. It should be understood in a preferred embodiment of the present invention that the tattoo ink applicator employs a round shader tattoo needle configuration in order to achieve desired application of the phosphorescent tattoo ink formulation 100. It should be understood within the scope of the present invention that alternate needle types and configurations could be utilized. While conventional particle sizes of pigment of tattoo ink ranges between 0.1-0.7 microns, the formulation of the present invention has a pigment base with a particle size of 1 micron.

In step 116, the dermal presentation of the phosphorescent tattoo ink formulation 100 is executed with the applicator employing the round shader tattoo needle configuration. Step 118, the tattoo created from the phosphorescent tattoo ink formulation 100 has light presented thereto either naturally or via artificial techniques. In step 120, the tattoo created with the phosphorescent tattoo ink formulation 100 emits a phosphorescence as a result of the pigment base forming a part of the phosphorescent tattoo ink formulation 100. It is further contemplated within the scope of the present invention that the pigment base could have introduced thereinto an amount of zinc sulfide. Furthermore, it should be understood within the scope of the present invention that the formulation of the present invention includes no more than thirty-three percent of the pigment base (strontium aluminate) and can be comprised of a percentage less than thirty-three percent.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding description is, therefore, not intended to be limited to the specific forms set forth herein, but on the contrary, it is intended to cover such alternatives, modifications, and equivalents, as can be reasonably included within the spirit and scope of the invention.

What is claimed is:

1. A method of formulating a tattoo ink wherein the tattoo ink formulation is operable to be phosphorescent subsequent dermal presentation wherein the method comprises the steps of:
selecting a pigment base, wherein the pigment base of the tattoo ink formulation is strontium aluminate;
choosing a dopant, wherein the dopant is either europium or dysprosium;
doping the pigment base, wherein the pigment base is doped with the dopant;
measuring a desired amount of glycerin, wherein a volume of glycerin is measure employing appropriate equipment;
thinning the glycerin, wherein the glycerin is thinned so as to reduce the viscosity thereof;
mixing the glycerin and the pigment base, wherein the glycerin and pigment base are mixed utilizing suitable equipment;
loading a tattoo needle, wherein the mixture of the formulation of the present invention is loaded into a tattoo needle;
presenting the formulation dermally, wherein the tattoo needle is utilized to execute dermal presentation of the formulation to create a tattoo;
exposing the tattoo to light;
emitting a phosphorescence light, wherein the tattoo emits a phosphorescence light.

2. The method of formulating the tattoo ink wherein the tattoo ink formulation is operable to be phosphorescent subsequent dermal presentation as recited in claim 1, wherein the glycerin in thinned utilizing ethanol and wherein the ethanol comprises approximately one to two percent of the volume of the formulation.

3. The method of formulating the tattoo ink wherein the tattoo ink formulation is operable to be phosphorescent subsequent dermal presentation as recited in claim 2, wherein the pigment base does not exceed thirty three percent of a total volume of the formulation.

4. The method of formulating the tattoo ink wherein the tattoo ink formulation is operable to be phosphorescent subsequent dermal presentation as recited in claim 3, wherein the dermal presentation is executed utilizing a round shader tattoo needle.

5. The method of formulating the tattoo ink wherein the tattoo ink formulation is operable to be phosphorescent subsequent dermal presentation as recited in claim 4, wherein the pigment base has a particle size of one micron.

6. The method of formulating the tattoo ink wherein the tattoo ink formulation is operable to be phosphorescent subsequent dermal presentation as recited in claim 5, and further including the step of storing the formulation at a temperature above ten degrees Celsius.
